(19) United States
(12) Patent Application Publication
(10) Pub. No.: US 2017/0342376 A1
(43) Pub. Date: Nov. 30, 2017

(54) FIBER STRUCTURE FOR USE AS CELL SCAFFOLD MATERIAL

(71) Applicant: Toray Industries, Inc., Tokyo (JP)

(72) Inventors: Koji Kadowaki, Otsu-shi (JP); Chisa Kuga, Otsu-shi (JP); Masaki Fujita, Otsu-shi (JP); Kazuhiro Tanahashi, Otsu-shi (JP); Satoshi Yamada, Otsu-shi (JP); Takayuki Kaneko, Otsu-shi (JP); Hiroshi Tsuchikura, Otsu-shi (JP)

(21) Appl. No.: 15/521,985

(22) PCT Filed: Oct. 30, 2015
(86) PCT No.: PCT/JP2015/080675

§ 371 Date: Apr. 26, 2017

(30) Foreign Application Priority Data
Oct. 31, 2014 (JP) 2014-222967

(51) Int. Cl. C12N 5/00; A61L 27/50; A61L 27/18; D01F 6/62; D03D 15/00; D03D 1/00

(52) U.S. Cl. CPC C12N 5/0068; D03D 15/0088; D03D 1/00; A61L 27/18; D01F 6/62; A61L 27/50; C12N 2533/30

(57) ABSTRACT

A fiber structure can be used as a cell scaffold material, which fiber structure includes a multifilament formed by bundling monofilaments having an average fiber diameter of 1 to 15 μm, wherein each of the monofilaments satisfies Formula (1): (Y/X)×100>50 . . . (1) wherein, in Formula (1), X represents the number of monofilaments for which the average crossing angle is investigated, and Y represents the number of monofilaments having an average crossing angle of not more than 25° in X.

### TECHNICAL FIELD

This disclosure relates to a fiber structure which can be used as a cell scaffold material.

### BACKGROUND

Base materials using various materials have been conventionally developed as base materials to support cells thereon during cell culturing. For example, in general cell culturing, surface-hydrophilized polystyrene (hereinafter referred to as “PS”) plastic culture dishes and glass culture dishes are used. Cells are cultured by allowing their adhesion, spreading, and growth on such culture dishes.

On the other hand, it is known that, since the culture environment during culturing on a plastic culture dish or a glass culture dish is largely different from an in vivo environment, material properties such as chemical properties, shape, and mechanical properties of the material surface may influence adhesion, spreading, growth, migration, and differentiation of cells during their culturing. In view of this, studies have been carried out for development of cell scaffold materials using various materials for the purpose of controlling material properties such as chemical properties, shape, and mechanical properties of the material surface.

In particular, since the actual extracellular matrix is known to be constituted of micron-sized skeletons and nano-sized fibers, a number of cell scaffold materials using fiber structures such as non-woven fabrics, woven fabrics, and knitted fabrics as materials have been developed to mimic in vivo structures.

For example, in terms of the cell scaffold materials using non-woven fabrics, preparation of a non-woven fabric composed of polylactic acid using a method called the electrospinning method, wherein a solution containing a fiber-forming substance composed of a highly bioavailable polylactic acid is introduced into an electric field, and the liquid is drawn toward an electrode, thereby forming fibers, has been disclosed. It is reported that use of the electrospinning method enables preparation of a non-woven fabric having a smooth surface with a micron-sized fiber diameter, and that improvement of the cellular adhesiveness is possible by the micron-sized non-woven fabric (JP 2004-290133 A and JP 2012-192105 A).

It is also reported that, when a non-woven fabric is prepared by the electrospinning method, uniformity of the surface properties, fiber diameter, and fiber orientation of the non-woven fabric influences the cellular adhesiveness and the like (A. Hadjizadeh et al., Journal of Biomedical Nanotechnology, 2013, Vol. 9(7), p. 1195).

In terms of the cell scaffold materials using knitted fabrics, formation of a tubular body by alternatively knitting fiber bundles prepared by bundling a plurality of ultrafine fibers having a diameter of about 1 to 50 μm composed of polylactic acid, and use of the tubular body after coating of its outer surface with collagen as a scaffold for induction of regeneration and growth of nerve cells, have been reported (JP 2009-153947 A).

In terms of the cell scaffold materials using woven fabrics, use of a woven fabric of polyester ultrafine fibers having a fiber fineness of not more than 1.0 denier as a cell scaffold material has been reported (WO 88/002398). It is reported that this woven fabric of ultrafine fibers is prepared by weaving composite fibers using polyester as an island component, and PS as a sea component, and then performing sea removal treatment to remove the sea component PS, thereby allowing formation of a woven fabric of polyester ultrafine fibers, followed by fluffing the tissue surface of the ultrafine fibers to increase the cell-contacting area, to thereby improve the cellular adhesiveness.

As a method of improving chemical properties of the material surface, a method in which the surface of ultrafine fibers of not more than 1.0 denier is subjected to plasma treatment, or sulfone groups and/or carboxyl groups are given to the surface to give anionic hydrophilic properties to the surface, to thereby increase the cell affinity and hence to improve the cell growth capacity (JP 01-034276 A).

However, although JP '133, JP '105, JP '947, WO '398 and JP '276 describe improvement of cell culture efficiency, especially adhesiveness, by changing physical properties of fibers such as the fiber diameter, or by changing chemical properties of the fiber surface, they do not describe improvement of the adhesiveness as well as the growth capacity of cells by controlling both the orientation of fibers constituting the fiber bundle and the average fiber diameter.

The cell culture base material used in A. Hadjizadeh et al. is a non-woven fabric composed of monofilaments. Therefore, although its fiber diameter can be controlled, its fiber orientation cannot be sufficiently uniform.

Although WO '398 describes improvement of the cellular adhesiveness by increasing the cell-contacting area by fluffing of the tissue surface of the ultrafine fibers, the orientation of the fibers constituting the fiber bundle cannot be uniform in cases where the fluffing is carried out.

Although there are methods such as the method in JP '276, in which the cell growth capacity is improved by chemical modification of the fiber surface, an additional process is required for the chemical modification in cases where chemical modification and the like are carried out, which is problematic.

In view of this, it could be helpful to provide a fiber structure that can be used as a cell scaffold material exhibiting improvement in both the cellular adhesiveness and the cell growth capacity, which improvement is achieved by controlling the fiber orientation in the multifilament and the average fiber diameter, which are physical properties.

### SUMMARY

We thus provide:

(1) A fiber structure which can be used as a cell scaffold material, the fiber structure comprising a multifilament formed by bundling monofilaments having an average fiber diameter of 1 to 15 μm, wherein each monofilament in the multifilament satisfies the condition of Formula (1):

(Y/X)×100>50 (1)

wherein in Formula (1), X represents the number of monofilaments for which the average crossing angle is investigated, and Y represents the number of monofilaments having an average crossing angle of not more than 25° in X.
(2) The fiber structure according to (1), which is a woven fabric.
(3) The fiber structure according to (1) or (2), wherein the monofilament arranged on the surface of the multifilament is a monofilament containing a polymer selected from the group consisting of polyester, polypropylene, acryl, polyamide, polystyrene, polyvinyl chloride, polyurethane, polysulfone, polyethersulfone, and polymethyl methacrylate.
(4) The fiber structure according to (3), wherein the monofilament arranged on the surface of the multifilament is a monofilament composed of polyethylene terephthalate or polybutylene terephthalate.
(5) The fiber structure according to any one of (1) to (4), wherein the cross-sectional shape of the monofilament is a flat multilobed shape with six to ten lobes.
(6) A cell scaffold comprising the fiber structure according to any one of (1) to (5).
(7) A cell scaffold for medical use, comprising the fiber structure according to any one of (1) to (5).

The fiber structure which can be used as a cell scaffold material improves both the cellular adhesiveness and the cell growth capacity since both the orientation of monofilaments in the multifilament and the average fiber diameter are controlled so that use of the fiber structure as an excellent cell scaffold material is possible.

### DETAILED DESCRIPTION

The fiber structure that can be used as a cell scaffold material is characterized in that it comprises a multifilament formed by bundling monofilaments having an average fiber diameter of 1 to 15 μm, wherein each monofilament in the multifilament satisfies Formula (1):

(Y/X)×100>50 (1)

wherein, in Formula (1), X represents the number of monofilaments for which the average crossing angle is investigated, and Y represents the number of monofilaments having an average crossing angle of not more than 25° in X.

Examples are described below, but this disclosure is not limited to these examples. The following terms are defined as described below unless otherwise specified.

“Cell scaffold” means a base material used to culture cells in vivo or in vitro, and “cell scaffold material” means a material to be used as a cell scaffold.

The cell scaffold material may be used to culture any cells in vivo or in vitro. The cell scaffold material is preferably used to culture adherent cells from the viewpoint of better exertion of the action to immobilize cells by adhesion.

“Multifilament” means a fiber bundle formed by bundling a plurality of monofilaments, and “monofilaments having an average crossing angle of not more than 25°” means monofilaments constituting a multifilament that are crossed with each other and have an average crossing angle S of not more than 25°, or monofilaments constituting a multifilament that are not crossed with each other (average crossing angle S=0°).

“Average crossing angle S” means a value determined by arbitrarily selecting a multifilament from a fiber structure, focusing on positions where monofilaments in the multifilament are crossed with their adjacent monofilaments based on observation of a photograph at a magnification of ×400 (viewing area, about 0.48 mm²), choosing the three positions having the largest crossing angles, and calculating the average value of the three crossing angles. Two angles are formed when two monofilaments are crossed with each other. The crossing angle corresponds to the smaller angle, that is, the angle having a value of 0° to 90°. When no position in a multifilament is found to have a crossing angle of not less than 25°, the monofilaments constituting the multifilament are regarded as being not crossed with each other (average crossing angle S=0°).

When the average crossing angle S between the monofilaments constituting the multifilament is not less than 25°, the monofilaments have different orientations so that the cellular adhesiveness and the cell growth capacity decrease. To achieve a uniform monofilament orientation, the woven fabric is preferably produced such that the fiber direction is not disturbed by, for example, yarn breakage or fluffing in the multifilament, and such that steps by application of an external force such as fabric raising, loop formation, and water jet punching to the multifilament portion are avoided. The average crossing angle S is most preferably 0° from the viewpoint of the monofilament orientation.

The ratio of monofilaments having an average crossing angle S of not more than 25° in the multifilament is calculated according to Formula (1). A sample was equally divided into four portions such that the angle of the intersection was 90°, and the average crossing angle was measured for 10 monofilaments in each portion (a total of 40 monofilaments) (the measurement was carried out for three positions per monofilament, that is, a total of 120 positions). The ratio was calculated according to Formula (1):

(Y/X)×100>50 (1)

wherein, in Formula (1), X represents the number of monofilaments for which the average crossing angle was investigated, and Y represents the number of monofilaments having an average crossing angle of not more than 25° in X.

In Formula (1), the value of (Y/X)×100 is preferably not less than 50, more preferably 100. When the value of (Y/X)×100 is less than 50, inhibition of the growth of cells along the orientation occurs, leading to a decrease in the cell growth capacity, which is not preferred.

“Average fiber diameter” is a value determined by observing cross sections of monofilaments in the multifilament at arbitrary 10 positions using a scanning electron microscope (manufactured by Hitachi High-Technologies Corporation), and calculating the average of their diameters. When the monofilament has a flat six- to ten-lobed shape, “average fiber diameter” is a value calculated by averaging the long diameter A and the short diameter B, wherein the long diameter A is the longest diameter connecting apexes on the circumcircle of the flat multilobed shape, and the short diameter B is the longest short diameter among the short diameters corresponding to the diameters connecting the apexes of the protruded portions of the flat multilobed shape.

The average fiber diameter of the multifilament is preferably 1 to 15 μm, more preferably 1 to 10 μm. When the average fiber diameter is not more than 1 μm or not less than 10 μm, the cellular adhesiveness decreases, which is not preferred.

Preferred specific examples of the fiber structure include non-woven fabrics, woven fabrics, knitted fabrics, tubes, and meshes. Woven fabrics are more preferred.

Although the multifilament may be used for one of the warp and the weft in the woven fabric, the multifilament is preferably used for both the warp and the weft. The “surface of the multifilament” is composed of the monofilaments exposed on the surface among the monofilaments forming the multifilament. The “surface of the multifilament” also includes monofilaments only partially exposed on the surface.

Although the type of the monofilaments constituting the multifilament is not limited, the monofilaments arranged on the surface of the multifilament are preferably monofilaments composed of a polymer selected from the group consisting of polyester, polypropylene, nylon, acryl, polyamide, and PS. From the viewpoint of the cost, cellular adhesiveness, and cell growth capacity, the monofilaments are preferably those composed of polyester.

The monofilaments composed of polyester are preferably monofilaments composed of polyethylene terephthalate, polybutylene terephthalate, or nylon. The monofilaments are more preferably composed of polyethylene terephthalate or polybutylene terephthalate.

The cross-sectional shape of the monofilament constituting the multifilament is not limited. Examples of the cross-sectional shape include known cross-sectional shapes such as circular, triangular, flat, and hollow shapes. The cross-sectional shape is preferably a flat multilobed shape with six to ten lobes, more preferably a flat eight-lobed shape.

“The cross-sectional shape of the monofilament is a flat multilobed shape with six to ten lobes” means that the monofilament has a cross-sectional shape which satisfies Formulae (2) to (5) at the same time:

Degree of flatness (A/B)=1.2 to 2.2 (2)

Degree of deformation I (C/D)=1.1 to 1.3 (3)

Degree of flatness II (B/D)=1.1 to 1.6 (4)

A>B>C>D (5)

wherein A represents the long diameter A, which is the longest diameter connecting apexes on the circumcircle of the flat multilobed shape; B represents the short diameter B, which is the longest short diameter perpendicular to the long diameter A among the short diameters that correspond to the diameters connecting the apexes of the protruded portions of the flat multilobed shape; C represents the short diameter C, which is the same as the short diameter B or the second longest short diameter; and D represents the short diameter D, which is the shortest short diameter among the short diameters connecting the bottom points of the recessed portions of the flat multilobed shape.

The fiber structure is preferably used in vitro as a cell scaffold. More specifically, the fiber structure is preferably used as a base material for use in culturing cells in vitro.

The fiber structure is preferably used as a cell scaffold for medical use. More specifically, the fiber structure is more preferably used for medical equipment for implanting to be embedded in the body such as artificial blood vessels and stent-grafts.

### EXAMPLES

Fiber structures and scaffolds are described below in detail by way of Examples and Comparative Examples. However, this disclosure is not limited thereto. The monofilament fineness in each of Examples and Comparative Examples is calculated according to the procedure of JIS L 1013 (2010) 8.3.1 A, wherein the fineness based on the corrected weight is measured at a predetermined load of 0.045 cN/dtex to provide the total fineness, and the resulting total fineness is divided by the number of monofilaments.

### Example 1

A woven fabric constituted of warp and weft yarns composed of multifilaments having a monofilament fineness of about 2.33 dtex and a total fineness of 84 dtex, wherein each multifilament is constituted of monofilaments composed of polyester fibers and having a circular cross-sectional shape, was prepared to provide a fiber structure 1. As a result of evaluation of the average fiber diameter using a scanning electron microscope (manufactured by Hitachi High-Technologies Corporation), the average fiber diameter was found to be 15 μm. As a result of measurement of the average crossing angle S using a microscope VHX-2000 (manufactured by Keyence Corporation), the value of Formula (1), (Y/X)×100, was found to be 100 in the fiber structure 1. The fiber structure 1 prepared was sterilized with ethylene oxide gas (hereinafter referred to as “EOG”), and subjected to tests for the cell growth capacity and the cellular adhesiveness. The results are shown in Table 1.

### Example 2

A woven fabric constituted of warp and weft yarns composed of multifilaments having a monofilament fineness of about 0.306 dtex and a total fineness of 44 dtex, wherein each multifilament is constituted of monofilaments composed of polyester fibers and having a circular cross-sectional shape, was prepared to provide a fiber structure 2. As a result of evaluation of the average fiber diameter using a scanning electron microscope (manufactured by Hitachi High-Technologies Corporation), the average fiber diameter was found to be 5 μm. As a result of measurement of the average crossing angle S using a microscope VHX-2000 (manufactured by Keyence Corporation), the value of Formula (1), (Y/X)×100, was found to be 100 in the fiber structure 2. The fiber structure 2 prepared was sterilized with EOG, and subjected to tests for the cell growth capacity and the cellular adhesiveness. The results are shown in Table 1.

### Example 3

A woven fabric constituted by warp and weft yarns composed of multifilaments having a monofilament fineness of about 0.0838 dtex and a total fineness of 52.8 dtex, wherein each multifilament is constituted of monofilaments composed of polyester fibers and having a circular cross-sectional shape, was prepared to provide a fiber structure 3. As a result of evaluation of the average fiber diameter using a scanning electron microscope (manufactured by Hitachi High-Technologies Corporation), the average fiber diameter was found to be 3 μm. As a result of measurement of the average crossing angle S using a microscope VHX-2000 (manufactured by Keyence Corporation), the value of Formula (1), (Y/X)×100, was found to be 100 in the fiber structure 3. The fiber structure 3 prepared was sterilized with EOG, and subjected to tests for the cell growth capacity and the cellular adhesiveness. The results are shown in Table 1.

### Example 4

A woven fabric constituted by warp and weft yarns composed of multifilaments having a monofilament fineness of about 0.0125 dtex and a total fineness of 56 dtex, wherein each multifilament is constituted by monofilaments composed of polyester fibers and having a circular cross-sectional shape, was prepared to provide a fiber structure 4. As a result of evaluation of the average fiber diameter using a scanning electron microscope (manufactured by Hitachi High-Technologies Corporation), the average fiber diameter was found to be 1 μm. As a result of measurement of the average crossing angle S using a microscope VHX-2000 (manufactured by Keyence Corporation), the value of Formula (1), (Y/X)×100, was found to be 100 in the fiber structure 4. The fiber structure 4 prepared was sterilized with EOG, and subjected to tests for the cell growth capacity and the cellular adhesiveness. The results are shown in Table 1.

### Example 5

A woven fabric constituted by warp and weft yarns composed of multifilaments having a monofilament fineness of about 0.0838 dtex and a total fineness of 52.8 dtex, wherein each multifilament is constituted of monofilaments composed of polyester fibers and having a circular cross-sectional shape, was prepared. As a result of evaluation of the average fiber diameter using a scanning electron microscope (manufactured by Hitachi High-Technologies Corporation), the average fiber diameter was found to be 3 μm. A half portion of the prepared woven fabric was fluffed using abrasive paper, and the border section was punched out using a puncher such that the value of Formula (1), (Y/X)×100, became 50, to provide a fiber structure 5. As a result of measurement of the average crossing angle S using a microscope VHX-2000 (manufactured by Keyence Corporation), the value of Formula (1), (Y/X)×100, was found to be 50 in the fiber structure 5. The fiber structure 5 prepared was sterilized with EOG, and subjected to tests for the cell growth capacity and the cellular adhesiveness. The results are shown in Table 1.

### Example 6

A woven fabric constituted by warp and weft yarns composed of multifilaments having a monofilament fineness of about 1.56 dtex and a total fineness of 56 dtex, wherein each multifilament is constituted by monofilaments composed of polyester fibers and having a flat eight-lobed cross-sectional shape, was prepared to provide a fiber structure 6. As a result of evaluation of the average fiber diameter using a scanning electron microscope (manufactured by Hitachi High-Technologies Corporation), the average fiber diameter was found to be 12 μm. As a result of measurement of the average crossing angle S using a microscope VHX-2000 (manufactured by Keyence Corporation), the value of Formula (1), (Y/X)×100, was found to be 100 in the fiber structure 6. The fiber structure 6 prepared was sterilized with EOG, and subjected to tests for the cell growth capacity and the cellular adhesiveness. The results are shown in Table 1.

### Comparative Example 1

A woven fabric constituted by warp and weft yarns composed of multifilaments having a monofilament fineness of about 5.6 dtex and a total fineness of 84 dtex, wherein each multifilament is constituted by monofilaments composed of polyester fibers and having a circular cross-sectional shape, was prepared to provide a fiber structure 7. As a result of evaluation of the average fiber diameter using a scanning electron microscope (manufactured by Hitachi High-Technologies Corporation), the average fiber diameter was found to be 23 μm. As a result of measurement of the average crossing angle S using a microscope VHX-2000 (manufactured by Keyence Corporation), the value of Formula (1), (Y/X)×100, was found to be 100 in the fiber structure 7. The fiber structure 7 prepared was sterilized with EOG, and subjected to tests for the cell growth capacity and the cellular adhesiveness. The results are shown in Table 1.

### Comparative Example 2

A woven fabric constituted by warp and weft yarns composed of multifilaments having a monofilament fineness of about 0.00625 dtex and a total fineness of 56 dtex, wherein each multifilament is constituted of monofilaments composed of polyester fibers and having a circular cross-sectional shape, was prepared to provide a fiber structure 8. As a result of evaluation of the average fiber diameter using a scanning electron microscope (manufactured by Hitachi High-Technologies Corporation), the average fiber diameter was found to be 0.7 μm. As a result of measurement of the average crossing angle S using a microscope VHX-2000 (manufactured by Keyence Corporation), the value of Formula (1), (Y/X)×100, was found to be 100 in the fiber structure 8. The fiber structure 8 prepared was sterilized with EOG, and subjected to tests for the cell growth capacity and the cellular adhesiveness. The results are shown in Table 1.

### Comparative Example 3

A woven fabric constituted by warp and weft yarns composed of multifilaments having a monofilament fineness of about 0.0838 dtex and a total fineness of 52.8 dtex, wherein each multifilament is constituted of monofilaments composed of polyester fibers and having a circular cross-sectional shape, was prepared. As a result of evaluation of the average fiber diameter using a scanning electron microscope (manufactured by Hitachi High-Technologies Corporation), the average fiber diameter was found to be 3 μm. A three-quarter portion of the prepared woven fabric was fluffed using abrasive paper, and the border section was punched out using a puncher such that the value of Formula (1), (Y/X)×100, became 25, to provide a fiber structure 9. As a result of measurement of the average crossing angle S using a microscope VHX-2000 (manufactured by Keyence Corporation), the value of Formula (1), (Y/X)×100, was found to be 25 in the fiber structure 9. The fiber structure 9 prepared was sterilized with EOG, and subjected to tests for the cell growth capacity and the cellular adhesiveness. The results are shown in Table 1.

### Comparative Example 4

A woven fabric constituted by warp and weft yarns composed of multifilaments having a monofilament fineness of about 0.0838 dtex and a total fineness of 52.8 dtex, wherein each multifilament is constituted of monofilaments composed of polyester fibers and having a circular cross-sectional shape, was prepared. As a result of evaluation of the average fiber diameter using a scanning electron microscope (manufactured by Hitachi High-Technologies Corporation), the average fiber diameter was found to be 3 μm. The whole portion of the prepared woven fabric was fluffed using abrasive paper, and punched out using a puncher such that the value of Formula (1), (Y/X)×100, became 0, to provide a fiber structure 10. As a result of measurement of the average crossing angle S using a microscope VHX-2000 (manufactured by Keyence Corporation), the value of Formula (1), (Y/X)×100, was found to be 0 in the fiber structure 10. The fiber structure 10 prepared was sterilized with EOG, and subjected to tests for the cell growth capacity and the cellular adhesiveness. The results are shown in Table 1.

### Comparative Example 5

A woven fabric constituted of warp and weft yarns composed of multifilaments having a monofilament fineness of about 0.0125 dtex and a total fineness of 56 dtex, wherein each multifilament is constituted of monofilaments composed of polyester fibers and having a circular cross-sectional shape, was prepared. As a result of evaluation of the average fiber diameter using a scanning electron microscope (manufactured by Hitachi High-Technologies Corporation), the average fiber diameter was found to be 1 μm. The whole portion of the prepared woven fabric was fluffed using abrasive paper, and punched out using a puncher such that the value of Formula (1), (Y/X)×100, became 0, to provide a fiber structure 11. As a result of measurement of the average crossing angle S using a microscope VHX-2000 (manufactured by Keyence Corporation), the value of Formula (1), (Y/X)×100, was found to be 0 in the fiber structure 11. The fiber structure 11 prepared was sterilized with EOG, and subjected to tests for the cell growth capacity and the cellular adhesiveness. The results are shown in Table 1.

### Comparative Example 6

Polyethylene terephthalate pellets were dissolved in the mixed solvent of 1:1 of dichloromethane (Wako Pure Chemical Industries, Ltd.) and trifluoroacetic acid (Wako Pure Chemical Industries, Ltd.) for 24 hours with stirring. Using an electrospinning device, a non-woven fabric having an average fiber diameter of 2 μm was prepared to provide a fiber structure 12. As a result of evaluation of the average fiber diameter using a scanning electron microscope (manufactured by Hitachi High-Technologies Corporation), the average fiber diameter was found to be 2 μm. As a result of measurement of the average crossing angle S using a microscope VHX-2000 (manufactured by Keyence Corporation), the value of Formula (1), (Y/X)×100, was found to be 0 in the fiber structure 12. The fiber structure 12 was sterilized with EOG, and subjected to tests for the cell growth capacity and the cellular adhesiveness. The results are shown in Table 1.

### Example 7

A woven fabric constituted by warp and weft yarns composed of multifilaments having a monofilament fineness of about 1.56 dtex and a total fineness of 56 dtex, wherein each multifilament is constituted of monofilaments composed of polyester fibers and having a flat six-lobed cross-sectional shape, was prepared to provide a fiber structure 13. As a result of evaluation of the average fiber diameter using a scanning electron microscope (manufactured by Hitachi High-Technologies Corporation), the average fiber diameter was found to be 12 μm. As a result of measurement of the average crossing angle S using a microscope VHX-2000 (manufactured by Keyence Corporation), the value of Formula (1), (Y/X)×100, was found to be 100 in the fiber structure 13. The fiber structure 13 prepared was sterilized with EOG, and subjected to tests for the cell growth capacity and the cellular adhesiveness. The results are shown in Table 1.

### Example 8

A woven fabric constituted of warp and weft yarns composed of multifilaments having a monofilament fineness of about 1.56 dtex and a total fineness of 56 dtex, wherein each multifilament is constituted of monofilaments composed of polyester fibers and having a flat ten-lobed cross-sectional shape, was prepared to provide a fiber structure 14. As a result of evaluation of the average fiber diameter using a scanning electron microscope (manufactured by Hitachi High-Technologies Corporation), the average fiber diameter was found to be 12 μm. As a result of measurement of the average crossing angle S using a microscope VHX-2000 (manufactured by Keyence Corporation), the value of Formula (1), (Y/X)×100, was found to be 100 in the fiber structure 14. The fiber structure 14 prepared was sterilized with EOG, and subjected to tests for the cell growth capacity and the cellular adhesiveness. The results are shown in Table 1.

### Comparative Example 7

A woven fabric constituted of warp and weft yarns composed of multifilaments having a monofilament fineness of about 0.00114 dtex and a total fineness of 70 dtex, wherein each multifilament is constituted of monofilaments composed of polyester fibers and having a circular cross-sectional shape, was prepared to provide a fiber structure 15. As a result of evaluation of the average fiber diameter using a scanning electron microscope (manufactured by Hitachi High-Technologies Corporation), the average fiber diameter was found to be 0.3 μm. As a result of measurement of the average crossing angle S using a microscope VHX-2000 (manufactured by Keyence Corporation), the value of Formula (1), (Y/X)×100, was found to be 100 in the fiber structure 11. The fiber structure 15 prepared was sterilized with EOG, and subjected to tests for the cell growth capacity and the cellular adhesiveness. The results are shown in Table 1.

### Example 9

The fiber structure 2 prepared in Example 2 was used after sterilization with EOG, and the cell growth rate was evaluated. The results are shown in Table 2.

### Example 10

The fiber structure 3 prepared in Example 3 was used after sterilization with EOG, and the cell growth rate was evaluated. The results are shown in Table 2.

### Example 11

The fiber structure 7 prepared in Example 13 was used after sterilization with EOG, and the cell growth rate was evaluated. The results are shown in Table 2.

### Comparative Example 8

The fiber structure 8 prepared in Comparative Example 2 was used after sterilization with EOG, and the cell growth rate was evaluated. The results are shown in Table 2.

### Comparative Example 9

The fiber structure 10 prepared in Comparative Example 4 was used after sterilization with EOG, and the cell growth rate was evaluated. The results are shown in Table 2.

### Evaluation 1: Cell Growth Capacity/Cellular Adhesiveness Test

Each of the fiber structures 1 to 12 was punched into a disk sample having a diameter of 15 mm using a puncher. Each disk sample was placed in a well of a 24-well microplate for cell culture (manufactured by Sumitomo Bakelite Co., Ltd.) such that the inner-wall side faced upward, and a metal pipe-shaped weight having a thickness of 1 mm was placed on the top of the sample. To each well, normal human umbilical vein endothelial cells (Takara Bio Inc.) suspended in 2% FBS endothelial cell culture kit-2 (manufactured by Takara Bio Inc.) were added such that the well contained 5×10⁴ cells. The cells were cultured in 1 mL of a medium at 37° C. under an environment of 5% CO₂ for 48 hours. After rinsing the well with PBS(−) (manufactured by Nissui Pharmaceutical Co., Ltd.), 1 mL of a medium was added thereto, followed by addition of 100 μL of Cell Counting Kit-8 (manufactured by Dojindo Laboratories). The cells were then cultured at 37° C. under an environment of 5% CO₂ for 4 hours. Subsequently, the absorbance at 450 nm was measured using a microplate reader (MTP-300, manufactured by Corona Electric Co., Ltd.), followed by calculation of the absorbance as shown by Formula (6):

As=At−Ab (6)
At: measured absorbance
Ab: absorbance of the blank solution (the medium, and the solution of Cell Counting Kit-8; containing no cells)
As: calculated absorbance.

Since the amount of grown cells after the culture can be known from the calculated absorbance As, a score for the cell growth was determined based on the absorbance As. More specifically, when As was less than 0.3, the cell growth capacity was judged as being weak (+); when As was not less than 0.3 and less than 0.5, the cell growth capacity was judged as being moderate (++); and, when As was not less than 0.5, the cell growth capacity was judged as being strong (+++).

After fixing the cells in 10% formalin solution (manufactured by Wako Pure Chemical Industries, Ltd.), the shapes of adherent cells were observed using a scanning electron microscope (manufactured by Hitachi High-Technologies Corporation). The sample was equally divided into four portions, and observation of surfaces was carried out for each portion at a magnification of ×1000. The length of the minor axis and the length of the major axis were measured for not less than 30 cells in a total of 16 viewing areas where two or more cells are present, corresponding to 4 different viewing areas in each portion. According to Formula (7), the aspect ratio of each cell was calculated:

Ls=La/Lb (7)
La: length of the cell in the direction of the major axis
Lb: length of the cell in the direction of the minor axis
Ls: calculated aspect ratio of the cell.

Since adhesion of the cell can be judged based on the thus calculated aspect ratio Ls, the ratio of adherent cells to the observed cells, Rs, was calculated according to Formula (8). A score for the cell adhesiveness was then determined based on the calculated value. More specifically, when Rs was not more than 50, the cellular adhesiveness was judged as being weak (+); when Rs was more than 25 and less than 50, the cellular adhesiveness was judged as being moderate (++); and, when Rs was not more than 25, the cellular adhesiveness was judged as being strong (+++).

Rs (%)=(number of cells satisfying Ls<2)/(number of cells observed)×100 (8)

### Evaluation 2: Evaluation of Cell Growth Rate

Each of the fiber structures 2, 3, and 13 of Examples, and the fiber structures 8 and 10 of Comparative Examples, was punched into a disk sample having a diameter of 15 mm using a puncher. Each disk sample was placed in a well of a 24-well microplate for cell culturing (manufactured by Sumitomo Bakelite Co., Ltd.) such that the inner-wall side faced upward, and a metal pipe-shaped weight having a thickness of 1 mm was placed on the top of the sample. NIH3T3 cells suspended in Iscove's modified Dulbecco's medium (manufactured by Sigma-Aldrich) supplemented with 10% FBS were added to the well at 4×10⁴ cells/well. The cells were cultured in 1 mL of a medium at 37° C. under an environment of 5% CO₂ for 24, 48, or 72 hours. After rinsing with PBS(−) (manufactured by Nissui Pharmaceutical Co., Ltd.), 500 μL of a medium was added to the cells, and 20 μL of Cell Counting Kit-8 (manufactured by Dojindo Laboratories) was then added thereto, followed by performing culture at 37° C. under an environment of 5% CO₂ for 1 hour. Subsequently, the absorbance at 450 nm was measured using a microplate reader (MTP-300, manufactured by Corona Electric Co., Ltd.). The cell growth rates at Hour 48 and Hour 72 were calculated using Formula (9) and Formula (10) described below:

P₄₈=(A₄₈−Ab)/(A₂₄−Ab) (9)
P₄₈: cell growth rate at Hour 48
A₄₈: measured value of the absorbance at Hour 48 of the culture
A₂₄: measured value of the absorbance at Hour 24 of the culture
Ab: absorbance of the blank solution (the medium, and the solution of Cell Counting Kit-8; containing no cells)

P₇₂=(A₇₂−Ab)/(A₂₄−Ab) (10)
P₇₂: cell growth rate at Hour 72
A₇₂: measured value of the absorbance at Hour 72 of the culture
A₂₄: measured value of the absorbance at Hour 24 of the culture
Ab: absorbance of the blank solution (the medium, and the solution of Cell Counting Kit-8; containing no cells).

From the data shown in Table 1 and Table 2, it is clear that both the cell adhesiveness and the cell growth capacity can be improved by controlling the orientation of monofilaments in the multifilament and the average fiber diameter, which are physical properties.

TABLE 1

| | | | | | | Result of evaluation | |
|---|---|---|---|---|---|---|---|
| | | Average | | | | of cells | |
| | | fiber | | Fiber | Cross-sectional | Cell | Cellular |
| | (X/Y) × | diameter | Fiber | processing | shape of | growth | adhesive- |
| | 100 | (μm) | structure | method | monofilament | capacity | ness |
| Example 1 | 100 | 15 | Multifilament | Woven fabric | Circular shape | +++ | ++ |
| Example 2 | 100 | 5 | Multifilament | Woven fabric | Circular shape | +++ | +++ |
| Example 3 | 100 | 3 | Multifilament | Woven fabric | Circular shape | +++ | +++ |
| Example 4 | 100 | 1 | Multifilament | Woven fabric | Circular shape | +++ | +++ |
| Example 5 | 50 | 3 | Multifilament | Woven fabric | Circular shape | +++ | ++ |
| Example 6 | 100 | 12 | Multifilament | Woven fabric | Flat eight- | +++ | +++ |
| | | | | | lobed shape | | |
| Comparative | 100 | 23 | Multifilament | Woven fabric | Circular shape | ++ | ++ |
| Example 1 | | | | | | | |
| Comparative | 100 | 0.7 | Multifilament | Woven fabric | Circular shape | ++ | ++ |
| Example 2 | | | | | | | |
| Comparative | 25 | 3 | Multifilament | Woven fabric | Circular shape | ++ | + |
| Example 3 | | | | | | | |
| Comparative | 0 | 3 | Multifilament | Woven fabric | Circular shape | ++ | + |
| Example 4 | | | | | | | |
| Comparative | 0 | 1 | Multifilament | Woven fabric | Circular shape | + | + |
| Example 5 | | | | | | | |
| Comparative | 0 | 2 | Monofilament | Non-woven fabric | Circular shape | ++ | + |
| Example 6 | | | | | | | |
| Example 7 | 100 | 12 | Multifilament | Woven fabric | Flat six- lobed shape | +++ | +++ |
| Example 8 | 100 | 12 | Multifilament | Woven fabric | Flat ten-lobed shape | +++ | +++ |
| Comparative | 100 | 0.3 | Multifilament | Woven fabric | Circular shape | + | + |
| Example 7 | | | | | | | |

TABLE 2

| | | Average | | | | Cell growth rate | |
|---|---|---|---|---|---|---|---|
| | | fiber | | Fiber | Cross-sectional | At | At |
| | (X/Y) × | diameter | Fiber | processing | shape of | Hour | Hour |
| | 100 | (μm) | structure | method | monofilament | 48 | 72 |
| Example 9 | 100 | 5 | Multifilament | Woven fabric | Circular shape | 1.5 | 2.2 |
| Example 10 | 100 | 3 | Multifilament | Woven fabric | Circular shape | 1.7 | 2.7 |
| Example 11 | 100 | 12 | Multifilament | Woven fabric | Flat eight-lobed shape | 1.9 | 3.1 |
| Comparative | 100 | 0.7 | Multifilament | Woven fabric | Circular shape | 1.3 | 1.8 |
| Example 8 | | | | | | | |
| Comparative | 0 | 3 | Multifilament | Woven fabric | Circular shape | 1.2 | 1.4 |
| Example 9 | | | | | | | |

### INDUSTRIAL APPLICABILITY

The fiber structure which can be used as a cell scaffold material can be used as a cell scaffold material excellent in the cell adhesiveness and the cell growth capacity. The fiber structure can also be used by inclusion in a cell scaffold for medical use, especially for artificial blood vessels, stent-grafts and the like.

1-7. (canceled)

8. A fiber structure which can be used as a cell scaffold material, said fiber structure comprising a multifilament formed by bundling monofilaments having an average fiber diameter of 1 to 15 μm,
wherein each monofilament in said multifilament satisfies Formula (1):
(Y/X)×100≧50 (1)
wherein, in Formula (1), X represents the number of monofilaments for which the average crossing angle is investigated, and Y represents the number of monofilaments having an average crossing angle of not more than 25° in X.

9. The fiber structure according to claim 8, which is a woven fabric.

10. The fiber structure according to claim 8, wherein said monofilament is a monofilament containing a polymer selected from the group consisting of polyester, polypropylene, acryl, polyamide, polystyrene, polyvinyl chloride, polyurethane, polysulfone, polyethersulfone, and polymethyl methacrylate.

11. The fiber structure according to claim 10, wherein said monofilament is a monofilament composed of polyethylene terephthalate or polybutylene terephthalate.

12. The fiber structure according to claim 8, wherein the cross-sectional shape of said monofilament is a flat multilobed shape with six to ten lobes.

13. A cell scaffold comprising the fiber structure according to claim 8.

14. A cell scaffold for medical use, comprising the fiber structure according to claim 8.

15. The fiber structure according to claim 9, wherein said monofilament is a monofilament containing a polymer selected from the group consisting of polyester, polypropylene, acryl, polyimide, polystyrene, polyvinyl chloride, polyurethane, polysulfone, polyethersulfone, and polymethyl methacrylate.

16. The fiber structure according to claim 9, wherein the cross-sectional shape of said monofilament is a flat multilobed shape with six to ten lobes.

17. The fiber structure according to claim 10, wherein the cross-sectional shape of said monofilament is a flat multilobed shape with six to ten lobes.

18. The fiber structure according to claim 11, wherein the cross-sectional shape of said monofilament is a flat multilobed shape with six to ten lobes.

19. A cell scaffold comprising the fiber structure according to claim 9.

20. A cell scaffold comprising the fiber structure according to claim 10.

21. A cell scaffold comprising the fiber structure according to claim 11.

22. A cell scaffold comprising the fiber structure according to claim 12.

23. A cell scaffold for medical use, comprising the fiber structure according to claim 9.

24. A cell scaffold for medical use, comprising the fiber structure according to claim 10.

25. A cell scaffold for medical use, comprising the fiber structure according to claim 11.

26. A cell scaffold for medical use, comprising the fiber structure according to claim 12.
